(19) United States
(12) Patent Application Publication
(10) Pub. No.: US 2023/0312495 A1
(43) Pub. Date: Oct. 5, 2023

(54) NEW ISOBUTYRAMIDE DERIVATIVES, COSMETIC AND/OR DERMATOLOGICAL PREPARATIONS CONTAINING SAID COMPOUNDS, AND THEIR USE FOR THE PROPHYLAXIS AND TREATMENT OF SENSITIVE, IN PARTICULAR INFLAMED SKIN OR INFLAMMATORY SKIN CONDITIONS

(71) Applicant: BEIERSDORF AG, Hamburg (DE)

(72) Inventors: Ludger KOLBE, Dohren (DE); Joern Hendrik REUTER, Henstedt Ulzburg (DE); Ahmed KAMAL, Hamburg (DE); Judith SEIDEL, Berlin (DE)

(21) Appl. No.: 18/041,742

(22) PCT Filed: Aug. 2, 2021
(86) PCT No.: PCT/EP2021/071523

§ 371 Date: Feb. 15, 2023

(30) Foreign Application Priority Data
Aug. 19, 2020 (DE) 102020210535.9

(51) Int. Cl. C07D 277/46; A61K 8/49; A61Q 19/00

(52) U.S. Cl. CPC C07D 277/46; A61K 8/49; A61Q 19/008

(57) ABSTRACT

New isobutyramides.

The present invention relates in particular to N-(4-(2,4-dihydroxy-3-methylphenyl)thiazol-2-yl)isobutyramide and hydroxymethoxyphenylthiazole isobutyramide, but also derivatives thereof, to cosmetic and/or dermatological preparations containing these compounds, and to the use thereof for the prophylaxis and treatment of sensitive, in particular inflamed skin or inflammatory skin conditions in human skin.

Changes associated with the aging process, but also other intrinsic and extrinsic factors (e.g. damage to the skin barrier, exposure to UV light, irritating or allergenic substances, mechanical action e.g., by shaving) can lead to skin irritation. In the context of this application, skin irritation is understood to mean any change in the skin that triggers sensory discomfort in humans or animals and/or is characterized by a dry, reddened and/or inflamed complexion. Of course, the term sensory discomfort also includes conditions such as itching or pain. In particular, skin irritation can include phenomenologically different skin conditions: Sensitive skin, sensitive skin including sensitive scalp, vulnerable skin, dry skin, atopic and psoriatic skin, irritated skin, rosacea, inflamed skin, and skin changes in diabetes mellitus, which in each case have a higher degree of severity in reddening of the skin, so-called erythema.

The problem of “sensitive skin” affects a growing number of adults and children. It is now believed that up to 50% of the population have sensitive skin (L. Misery et al., Ann. Dermatol. Venereol. 2005, 132, 425-429). Sensitive skin refers to skin having a lowered threshold for irritants, such as hyperreactive and intolerant skin, but also atopic skin. A phenomenon called “stinging” can be observed in people with sensitive, delicate or vulnerable skin. Typical disturbing phenomena associated with the terms “stinging” or “sensitive skin” are skin reddening, tingling, prickling, tightening and burning of the skin and itching. They can be caused by stimulating environmental conditions such as massage, exposure to surfactants, exposure to other chemical substances such as lactic acid, weather influences such as heat, cold, drought, but also humidity, thermal radiation and UV radiation, from the sun for example, or psychological stress.

“Sensitive” scalps are also characterized by skin reddening, tingling, prickling, burning and itching. Triggers are, for example, soap, shampoos or other hair care products, surfactants, hard water having a high concentration of lime and/or mechanical strain. Erythema and hyperseborrhea (excessive production of sebum) of the scalp and also dandruff are often associated with the phenomena described.

Atopy, a familial hypersensitivity of the skin and mucous membranes to environmental substances, with an increased disposition to develop instantaneous hypersensitivity reactions (allergies) to substances from the natural environment, can be observed in about 10-20% of the population of industrialized countries, with an increasing tendency. Atopy is believed to be genetic. Atopy can manifest as atopic dermatitis. The skin barrier is damaged and the skin is often inflamed and itchy.

The erythematous effect of the ultraviolet part of solar radiation or artificial radiation on the skin is well known. Whereas rays having a wavelength of less than 290 nm (the so-called UVC range) are absorbed by the ozone layer in the earth's atmosphere, rays in the range between 290 nm and 320 nm, the so-called UVB range, cause erythema, simple sunburn or actual burns of varying severity. Erythematous skin symptoms also occur as side effects of certain skin diseases or irregularities, for example acne, bacterially induced skin inflammation, cutaneous reactions, dermographism, skin soreness in general, skin moles, erysipelas, shingles, frostbite or burns. Rosacea is a particular form of reddening of the skin. Anti-inflammatory preparations can therefore also be used to advantage in general to reduce disease-related skin redness and in particular rosacea, to suppress it over a longer period of time and to prevent its recurrence to a large extent.

Skin inflammation, which is accompanied by redness and itching, is also caused by insect bites.

Anti-inflammatory agents can therefore also help to reduce the consequences of an insect bite, such as reddening, wheals, itching and painful swelling of the skin, suppress them over a longer period of time and prevent them from recurring.

Erythemas also occur more frequently in the diaper area of small children, and even more so in infants (diaper dermatitis). Incontinence, a condition that is particularly prevalent in older age, is also often associated with erythema and skin reddening as a result of constant exposure to moisture and irritants (incontinence dermatitis).

Although a large number of active ingredients having a skin irritation-reducing effect are already being used in the technical areas mentioned, alternatives are still being sought.

In the cosmetic and pharmaceutical industry there is therefore a constant need for compositions having a skin irritation-reducing effect. When searching for appropriate anti-inflammatory compositions, it should be noted that the substances used in cosmetic and/or pharmaceutical products must also be
toxicologically harmless,
well tolerated by the skin,
stable (especially in common cosmetic and/or pharmaceutical formulations),
preferably faintly odorous or (largely) odorless,
preferably colorless and non-staining, and
inexpensive to produce (i.e. using standard processes and/or starting from standard precursors).

The search for suitable (active) substances that possess one or more of the properties mentioned to a sufficient degree is made more difficult for the person skilled in the art by the fact that there is no clear relationship between the chemical structure of a substance on the one hand and the anti-inflammatory activity and stability thereof on the other. Furthermore, there is no predictable correlation between the anti-inflammatory effect, toxicological safety, skin tolerance and/or stability.

It was therefore the object of the present invention to specify an anti-inflammatory active ingredient which preferably fulfills one or more of the aforementioned secondary conditions and particularly preferably all of the aforementioned conditions.

The present invention relates to the use of topical preparations for removing sebum from the skin, in particular for eliminating and clearing comedones, for preventing the formation of comedones, for the prophylaxis and treatment of mild forms of acne and for the prophylaxis and control of seborrhea.

Overproduction of the sebaceous glands and the possible resulting skin disorders that develop therefrom, such as sebum retention, formation of comedones in the area of the sebaceous glands, i.e. on the face (especially forehead, nose and chin) and on the upper back and as a result the various forms of acne, are a frequently encountered skin problem that has not yet been solved to a satisfactory extent.

Sebum is the secretion of the sebaceous gland. Sebaceous glands are bulges of the follicular epithelium and are therefore constituents of the follicle, with which they form a functional unit. They are holocrine glands, which means that most of the gland cell is converted into the secretion sebum. The cells of the sebaceous gland, which are constantly renewed from below, become fatty and disintegrate, and the skin sebum thus formed is emptied through the follicle openings onto the surface of the skin.

The skin sebum, according to G. Leonardy (J.Ft. Jellinek Kosmetologie, Zweck and Aufbau kosmetischer Praparate (cosmetology, purpose and structure of cosmetic preparations), Dr. Alfred Huthig-Verlag, Heidelberg-Mainz-Basel, third completely revised and significantly expanded edition 1976, pages 26 to 29), consists of mono-, di- and triglycerides (C₁₀-C₁₈), waxes (C₁₆-C₂₆), wax esters (C₂₈-C₃₈), normal saturated fatty acids (C₁₀-C₁₈), branched-chain saturated fatty acids (C₁₁-C₁₈), multibranched-chain saturated fatty acids (C₁₃-C₁₈), monounsaturated fatty acids (C₁₁-C₁₈), polyunsaturated fatty acids (C_{13,} C₁₅-C₁₇), sterols (cholesterol, 7-dehydrocholesterol, 7-hydroxycholesterol), branched and unbranched hydrocarbons (C₃₀-C₄₀), squalene and phospholipids.

Together with the watery secretion of the eccrine sweat glands, the lipids of the sebaceous gland form the so-called hydro-lipid film of the skin. This surface film is an emulsion, which can be a water-in-oil or an oil-in-water emulsion. It has the function of keeping the skin surface supple and of regulating the water content of the deeper skin layers. When the sebum is well hydrated, the water content is at least 10 to 20% by weight and the sebum is hydrophilic. When the hydrophilic-lipophilic balance of the surface film is disrupted and the water content decreases, the sebum changes and becomes hydrophobic. The flow of sebum from the sebaceous gland and follicle is impeded. This leads to congestion of the skin sebum in the follicle openings, which can then subsequently lead to comedones and inflammation of the follicles.

The change in the skin sebum and the initiation of the formation of comedones can have various causes. For example: external influences such as incorrect cleansing habits and care, comedogenic substances in cosmetics, weather influences, alkaline soaps, harsh detergents. Increased sebaceous gland secretion and the formation of comedones can also develop as a result of genetic factors and hormonal influences. Here, too, comedones, inflammation, pre-acne and acne with the after-effects thereof can result.

The incidence of skin damage due to impaired sebaceous gland function and sebaceous gland disorders is increasing, making the reduction/prevention of comedone formation an urgent concern. However, previous attempts to solve comedone formation as a causal problem have led to few satisfactory results.

In addition to manually squeezing out comedones, many other cleansing methods are known that attempt to remove comedones and prevent them from forming in a lasting manner. These include special soaps, skin peeling compositions and the like. Emollients and astringents are also used. In addition, attempts are being made to reduce the tendency to acne by adding drying, keratolytic, antiseborrheic and antibacterial active ingredients to cosmetic and pharmaceutical preparations, without causing irritation or drying out of the skin.

However, skin cleansing degreases the skin and removes moisture from it. In addition, soaps have the disadvantage that the water-insoluble calcium and magnesium salts of the higher fatty acids that form when the soaps are used in hard water form slimy deposits on the skin. These precipitates remain on the skin longer when poorly rinsed off, clog the follicle openings and may result in the formation of comedones. Therefore, mainly syndets (i.e., surfactants without soap character) in the form of washing creams or washing lotions are used for skin cleansing. Although these syndets do not form lime soaps, treatment with highly surface-active agents has a stronger degreasing and drying effect on the skin than soap. The more frequently products containing soap and surfactants are applied to the skin, the more clearly their adverse effects, namely degreasing and drying out of the skin through destruction of the hydro-lipid film, come to the fore. The reduction in comedones almost always leads to a decrease in the water content of the upper layers of the skin and to solid calculus formation in the sebaceous glands, which in turn can induce inflammation. However, lowering the moisture content of the skin is counterproductive for a nurturing removal of the comedones.

Seborrhea is a predisposition to increased function of the sebaceous glands. Both scalp and facial skin appear greasy. The composition of seborrheic sebum is altered compared to normal sebum. 3 stages of development of seborrhea are distinguished:
1. Mild cases of simple seborrhea, greasy after 8 days.
2. Oily seborrhea, already greasy after 2-3 days.
3. Irreversible form, no longer reversible. Seborrhea, where the hair looks like it has been bathed in grease after just one day.

The excessive secretion of the sebaceous glands can be triggered, inter alia, by androgenetic disorders and has an esthetic detrimental effect on the overall appearance of the hair. This disorder can also be the cause of hair loss. The precursor in each case is the seborrheic condition of the scalp. Vegetative disorders and improper care can worsen the complexion and hair condition. In seborrhea, the hair itself can also be dry due to disturbances in the keratin structure. Dry, damaged hair is often caused by external stress such as sun or chemical treatments. Blow-drying that is too hot or not properly caring for damaged hair can lead to harm.

The causes of greasy hair are in the human body and are hormonal. Each hair has its own sebaceous gland, which produces fat (sebum, skin sebum). Sebum production is hormonally controlled, and depending on the hormone sensitivity of the sebaceous gland, over- or underproduction may occur. The sebum itself has the function of keeping the scalp supple. It reaches the scalp from the sebaceous gland and only later the hairline. There it is normally absorbed by the hair shaft and remains invisible. When sebum is overproduced, the hair shaft is no longer able to absorb it. It becomes visible as a greasy film on the hair. The result is stringy, greasy, shiny hair.

Because sebaceous gland production is dependent on hormone balance, the problem of greasy hair cannot be solved in principle, because the sebaceous glands constantly produce fat. Consistent care and high-quality skin care products are still the best way to combat greasy hair.

Greasy hair has very tiresome effects. The hair becomes stringy again just a short time after washing and the hairstyle does not last.

Contrary to popular belief, it is only a rumour that washing too often makes hair greasy even faster. Mild shampoos for greasy hair ensure that excess grease is removed. Hair and scalp are supplied with sufficient moisture and balance the overproduction of the sebaceous glands.

Greasy hair and dandruff are among the most common hair problems. These abnormalities are due to a disorder in the activity of the sebaceous glands. When the sebaceous glands are overactive, it is called seborrhea. Two forms can be distinguished: the oily form (seborrhea oleosa) and the dry form (seborrhea sicca).

Seborrhea Oleosa:

Here, there is overactivity of the sebaceous glands, in which the sebaceous glands produce too much and too oily sebum. The skin therefore has a greasy sheen, and the hair is greasy and stringy again right down to the tips just 2 to 3 days after washing.

### Seborrhea Sicca:

This is also due to overactivity of the sebaceous glands, but the skin sebum is drier and has a firmer consistency. With the scales of the epidermis, it forms large, easily friable sebum scales. The scalp has a waxy sheen, the hair regreases only at the roots, the lengths and particularly the tips are dry and even brittle.

The treatment of seborrhea includes, first of all, regular and thorough washing of the head with special shampoos, which can be carried out as often as seems necessary. The washing should be combined with massage of the connective tissue, because this empties the sebaceous glands more, which delays the regreasing.

The object of the present invention is to provide a preparation which does not have the disadvantages of the known and previously used compositions, which at the same time has a sebum-dissolving, lipid-regulating and caring effect, removes the existing comedones without irritation, effectively prevents the degreasing and drying of the skin, prevents the formation of comedones and the development of acne cosmetics, and also improves existing acne and at the same time cares for the skin.

Surprisingly, it has been found that the objects are achieved by

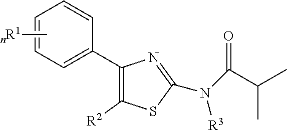

isobutyramides of the general formula
wherein
n: signifies that 1 to 5 identical and/or different substituents R¹ may be present, wherein the individual or plurality of substituents R¹: may each independently be: OH, CH₃, O—CH₃, O—(CO)—CH₃ (acetyl), where
R²: may be H or a C₁-C₄ alkyl chain, and where
R³: may be H or CH₃, but where
the isobutyramide of structure

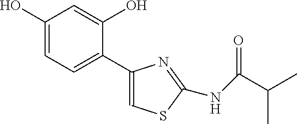 [SMILES: CC(C)C(=O)Nc1nc(-c2ccc(O)cc2O)cs1]

is excluded.

The isobutyramides according to the invention are excellent active ingredients for the prophylaxis and treatment of sensitive skin, itching and inflammatory phenomena in human skin.

Surprisingly, when the preparation is used, the comedogenic effect of the raw materials used in the preparation is also eliminated and therefore the formation of comedones and, accordingly, the development of acne is prevented.

It has also been found that the active ingredients used in accordance with the invention prevent the formation of seborrheic phenomena, particularly greasy hair, but also dandruff, and eliminate already existing seborrheic phenomena, particularly greasy hair, but also dandruff.

In addition, the present invention ensures very good skin care, combined with efficient removal of excess skin sebum.

Thus, according to the invention, a method for combating impure skin, acne or seborrheic phenomena, in particular greasy hair and/or dandruff, is also provided, characterized in that the active ingredients used according to the invention are brought into contact with the area affected by increased sebum production in a suitable cosmetic or dermatological carrier.

The prior art did not provide the slightest indication of the use according to the invention as an antiseborrheic active principle.

A further preferred embodiment of the present invention are therefore formulations to be used against dandruff, for example anti-dandruff shampoos.

EP 3 176 156 A1 describes similar isobutyramides, but the substances according to the invention are not specifically disclosed in this document.

Furthermore, the effect disclosed in EP 3 176 156 A1 is reduction in skin pigmentation. However, this effect does not occur with the isobutyramides (here) according to the invention and would also be undesirable according to the invention.

Cosmetic or dermatological preparations according to the invention preferably comprise 0.000001 to 10% by weight, particularly preferably 0.0001 to 3% by weight, and especially 30 preferably 0.001 to 1% by weight, of isobutyramides, based on the total weight of the preparations.

When applying the active ingredients used according to the invention or cosmetic or topical dermatological preparations having an effective content of active ingredients used according to the invention, an effective treatment of, but also prophylaxis for, deficient, sensitive or hypoactive skin conditions or deficient, sensitive or hypoactive conditions of skin appendages and/or inflammatory phenomena and/or itching is surprisingly possible.

It is particularly extremely advantageous according to the invention to use one or more isobutyramides according to the invention or cosmetic or topical dermatological preparations having an effective content of isobutyramides according to the invention for cosmetic or dermatological treatment or prophylaxis of undesirable skin conditions.

Advantageous isobutyramides in the context of the present invention are the following substances:

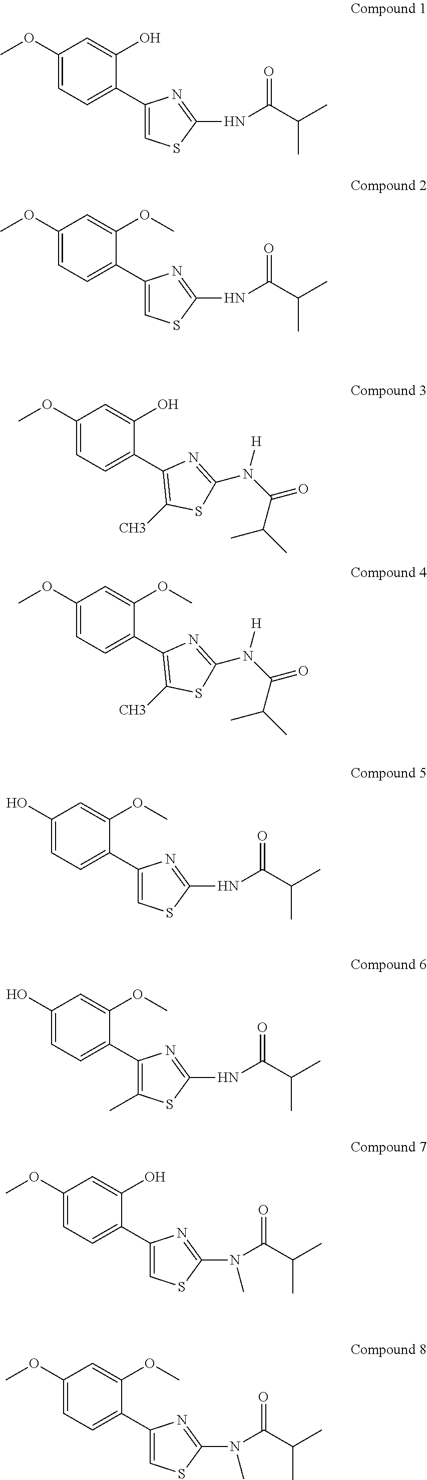

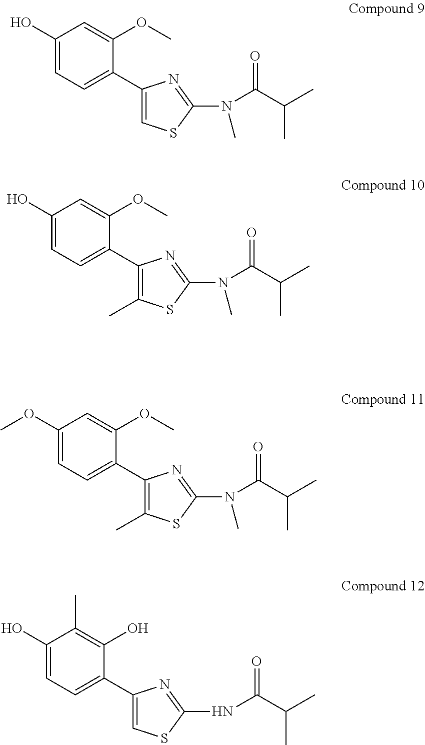

### N-(4-(2,4-Dihydroxy-3-Methylphenyl)Thiazol-2-Yl)Isobutyramide

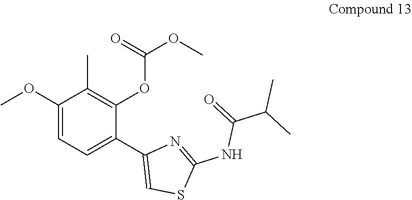 [SMILES: COC(=O)Oc1c(-c2csc(NC(=O)C(C)C)n2)ccc(OC)c1C]

The prophylaxis or the cosmetic or dermatological treatment with the active ingredient used according to the invention or with the cosmetic or topical dermatological preparations having an effective content of the active ingredient used according to the invention is effected in the usual manner, in such a way that the active ingredient used according to the invention or the cosmetic or topical dermatological preparations having an effective content of the active ingredient used according to the invention is applied to the affected areas of the skin.

The active ingredient used according to the invention can advantageously be incorporated into customary cosmetic and dermatological preparations, which can take various forms. For instance, they may for example be a solution, a water-in-oil (W/O) type emulsion or an oil-in-water (O/W) type emulsion, or multiple emulsions, for example of water-in-oil-in-water (W/O/W) type or oil-in-water-in-oil (O/W/O) type, a hydrodispersion or lipodispersion, a Pickering emulsion, a gel, a solid stick or even an aerosol.

Inventive emulsions in the context of the present invention, for example in the form of a cream, a lotion or a cosmetic milk, are advantageous and comprise, for example, fats, oils, waxes, and/or other fatty substances, and also water and one or more emulsifiers, as are customarily used for formulations of this type.

It is also possible and advantageous in the context of the present invention to include the active ingredient used according to the invention in aqueous systems or in surfactant preparations for cleansing the skin and the hair.

It is of course known to the person skilled in the art that sophisticated cosmetic compositions are generally not conceivable without the customary auxiliaries and additives. Examples of these include consistency regulators, fillers, fragrance, colorants, emulsifiers, additional active ingredients such as vitamins or proteins, light stabilizers, stabilizers, insect repellents, alcohol, water, salts, antimicrobial, proteolytic or keratolytic active substances, etc.

Mutatis mutandis, corresponding requirements apply to the formulation of medicinal preparations.

Topical medicinal compositions in the context of the present invention generally comprise one or more medicaments at an effective concentration. For the sake of simplicity and in order to make a clear distinction between cosmetic and medicinal use and corresponding products, reference is made to the statutory provisions of the Federal Republic of Germany (for example the Kosmetikverordnung [Cosmetics Ordinance] and Lebensmittel- und Arzneimittelgesetz [Food and Medicinal Products Act]).

Here it is also advantageous to include the active ingredient employed according to the invention as an additive in preparations that already contain other active ingredients for other purposes.

Correspondingly, cosmetic or topical dermatological compositions in the context of the present invention, depending on their structure, can be used, for example, as skin protection cream, cleansing milk, sunscreen lotion, nourishing cream, day or night cream, etc. It may be possible and advantageous to use the compositions according to the invention as a basis for pharmaceutical formulations.

For application, the cosmetic and dermatological preparations according to the invention are applied to the skin and/or hair in a sufficient amount in the manner customary for cosmetics.

Particularly preferred are those cosmetic and dermatological preparations which are in the form of a sunscreen. Advantageously, these can additionally comprise at least one further UVA filter and/or at least one further UVB filter and/or at least one inorganic pigment, preferably an inorganic micropigment.

In accordance with the invention, the cosmetic and dermatological preparations may comprise cosmetic auxiliaries such as are commonly used in such preparations, e.g. preservatives, bactericides, perfumes, substances to prevent foaming, dyes, pigments having a coloring effect, thickeners, moisturizing substances, fats, oils, waxes or other usual constituents of a cosmetic or dermatological formulation such as alcohols, polyols, polymers, foam stabilizers, electrolytes, organic solvents or silicone derivatives.

An additional content of customary antioxidants is generally preferred. According to the invention, favorable antioxidants which can be used are all antioxidants that are suitable or customary for cosmetic and/or dermatological uses.

The amount of aforementioned antioxidants (one or more compounds) in the preparations is preferably 0.001 to 30% by weight, particularly preferably 0.05-20% by weight, especially 1-10% by weight, based on the total weight of the preparation.

If the cosmetic or dermatological preparation in the context of the present invention is a solution or emulsion or dispersion, it is possible to use, as solvents:
water or aqueous solutions;
oils such as capric or caprylic acid triglycerides, but preferably castor oil;
fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids;
alcohols, diols or polyols of low carbon number, and also the ethers thereof, preferably ethanol, iospropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products.

In particular, mixtures of the abovementioned solvents are used. In the case of alcoholic solvents, water may be a further constituent.

If the cosmetic or dermatological preparation in the context of the present invention is a solution or emulsion or dispersion, it is possible to use, as solvents:
water or aqueous solutions;
oils such as capric or caprylic acid triglycerides, but preferably castor oil;
fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids;
alcohols, diols or polyols of low carbon number, and also the ethers thereof, preferably ethanol, iospropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products.

In particular, mixtures of the abovementioned solvents are used. In the case of alcoholic solvents, water may be a further constituent.

Suitable as propellant for cosmetic and/or dermatological preparations in the context of the present invention that are sprayable from aerosol containers are the readily volatile, liquefied propellants that are customary and known, for example hydrocarbons (propane, butane, isobutane), which can be used alone or in a mixture with one another. Compressed air may also be advantageously used.

A person skilled in the art is of course aware that there are inherently nontoxic propellants which would be suitable in principle for the realization of the present invention in the form of aerosol preparations, but which should nevertheless be dispensed with because of environmental unfriendliness or other concomitants, especially fluorinated hydrocarbons and chlorofluorocarbons (CFCs).

Cosmetic preparations in the context of the present invention can also be present as gels comprising not only an effective content of the active ingredient according to the invention and solvents customarily used therefor, preferably water, but also organic thickeners, for example gum arabic, xanthan gum, sodium alginate, cellulose derivatives, preferably methylcellulose, hydroxymethylcellulose, hydroxyethylcellulose, hydroxypropylcellulose, hydroxypropyl-methylcellulose, or inorganic thickeners, for example aluminum silicates such as bentonites, or a mixture of polyethylene glycol and polyethylene glycol stearate or distearate. The thickener is present in the gel, for example, in an amount between 0.1 and 30% by weight, preferably between 0.5 and 15% by weight.

It is advantageous according to the invention to use other oil-soluble UVA filters and/or UVB filters in the lipid phase and/or other water-soluble UVA filters and/or UVB filters in the aqueous phase in addition to the combinations according to the invention.

Advantageously, the light protection formulations may comprise further substances according to the invention which absorb UV radiation in the UVB range in order to provide cosmetic preparations which protect the skin from the entire range of ultraviolet radiation, in which the total amount of filter substances is, for example, from 0.1% to 30% by weight, preferably 0.5% to 10% by weight, especially 1% to 6% by weight, based on the total weight of the preparations. They can also serve as sunscreens.

### Proof of Efficacy:

The effect of the derivatives was investigated using a number of test methods, the relevance of which for cosmetic issues has been shown many times in recent years and which are described in detail by Kolbe et al. (Anti-inflammatory efficacy of Licochalcone A: correlation of clinical potency and in vitro effects. Kolbe L et al., Arch Dermatol Res. 2006 JunE;298(1):23-30). Prostaglandins (PG) are inflammatory mediators that can be rapidly formed from arachidonic acid and can increase redness and experience of pain. In the skin, reducing the release of PGE2 is particularly advised to prevent or reduce inflammation.

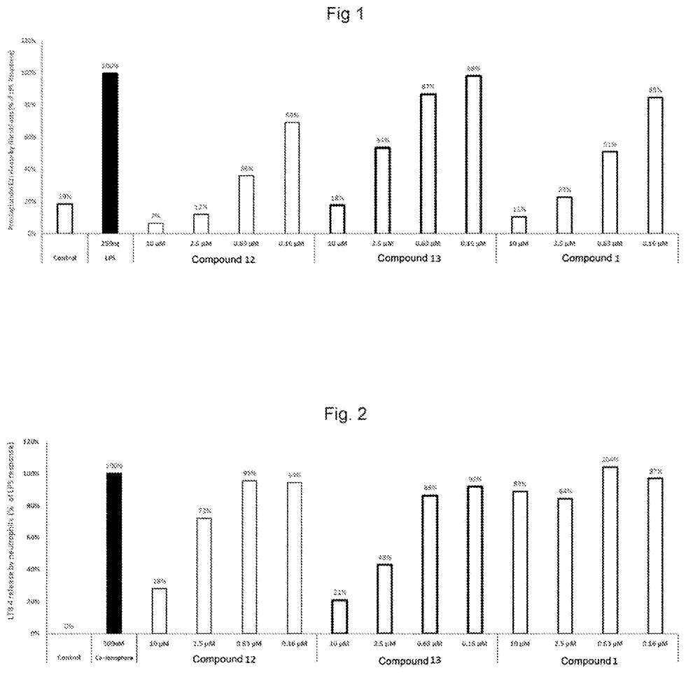

FIG. 1 demonstrates the reduction in LPS-stimulated PGE2 production in human dermal fibroblasts.

Leukotrienes, which also promote inflammation, can also be formed from arachidonic acid in the skin by migrating neutrophilic granulocytes. Therefore, a reduction in leukotrienes, especially LTB4, is also advised for efficient efficacy.

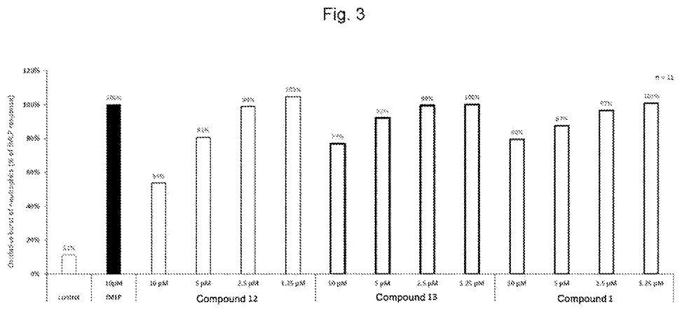

FIG. 2 demonstrates the reduction in Ca ionophore-stimulated LTB4.

Granulocytes are the first immune cells to migrate into the skin after an inflammatory stimulus. Although the “oxidative burst” of the granulocytes is very important for defence against infection, an excessive burst in the tissue can cause extensive collateral damage. In the case of severe inflammation, it may therefore be advisable to dampen this reaction with anti-inflammatory substances.

FIG. 3 demonstrates the reduction in the “oxidative burst” of fMLP-stimulated neutrophilic granulocytes.

It can be seen from FIGS. 1-3 that the isobutyramides claimed exhibit an extremely positive anti-inflammatory effect in the tests specified.

### Synthetic Methods for Isobutyramides Selected by Way of Example:

### Compound 1: N-(4-(2-Hydroxy-4-Methoxyphenyl)Thiazol-2-Yl)Isobutyramide

### Step 01

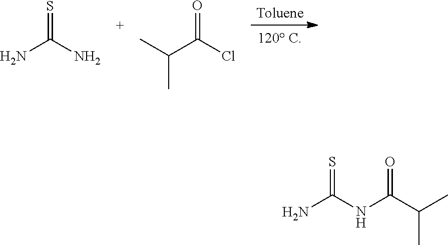

114 g (1.5 mol) of thiourea were initially charged in toluene (800 ml) and 80 g (0.75 mol) of isobutyryl chloride were added dropwise. The reaction solution was boiled under reflux for 3 hours, with 2 phases arising. The upper phase was decanted off and cooled. The precipitated white crystals were filtered off under suction and washed with toluene and dried under vacuum.

Yield: 62 g. ¹H-NMR (DMSO-D₆): 11.03 (bs, 1H), 9.66 (bs, 1H), 9.35 (bs, 1H), 2.72 (m, 1H), 1.03 (2, 6H) ppm;

### Step 02

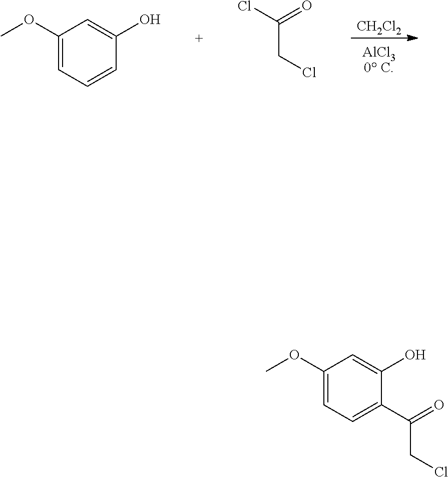

### Step 03

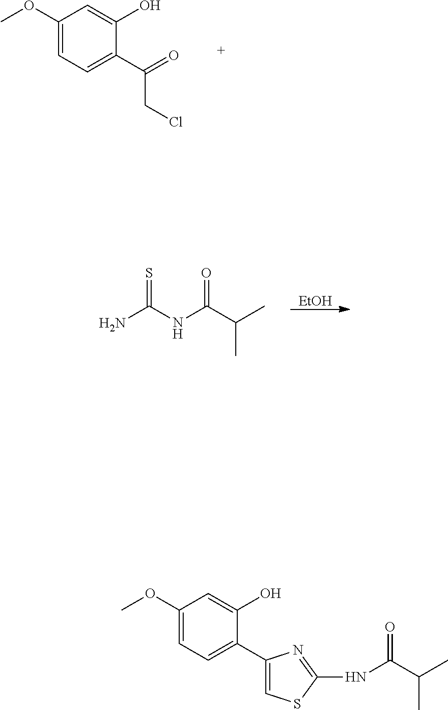

### Compound 2: N-(4-(2,4-Bis(Methoxy)Phenyl)Thiazol-2-Yl)Isobutyramide

### Step 01

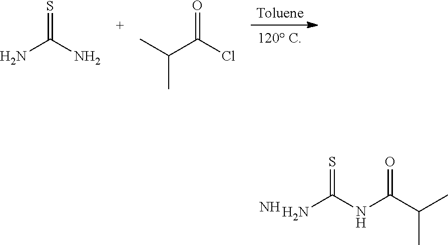

### Step 02

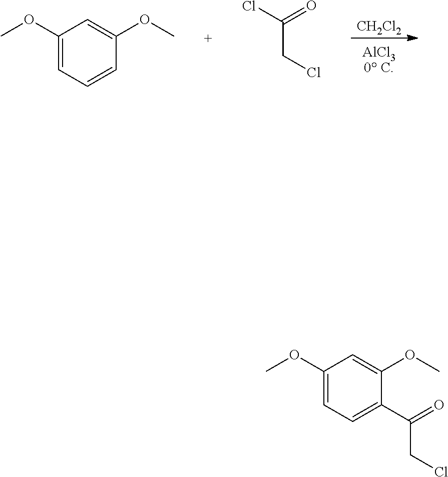

### Step 03

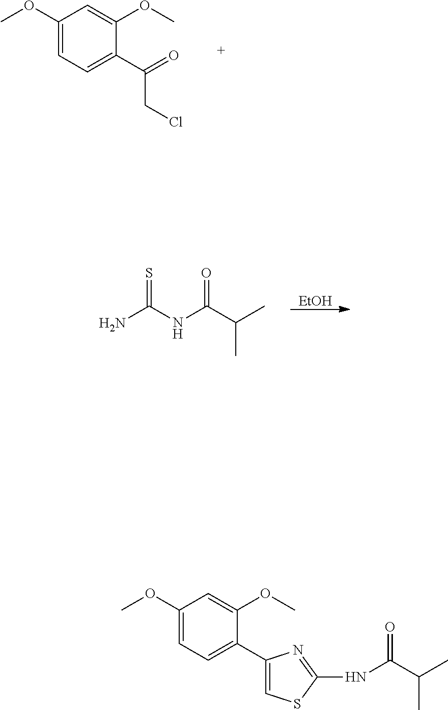

### Compounds 12 and 13

### Step 01

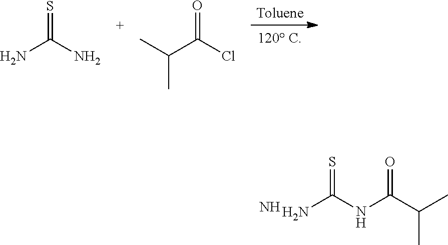

### Step 02

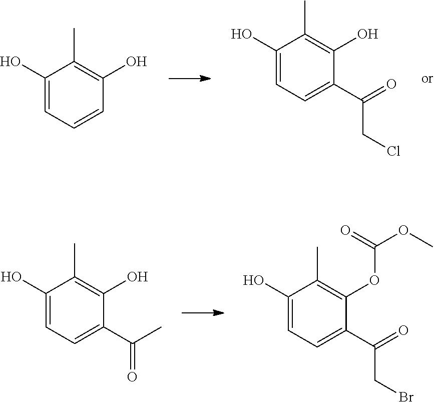

### Step 03

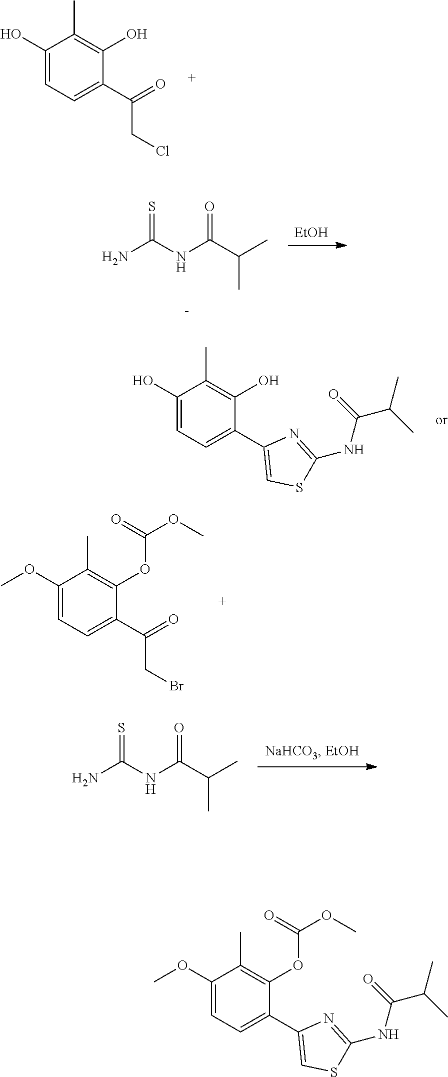

### Step 04

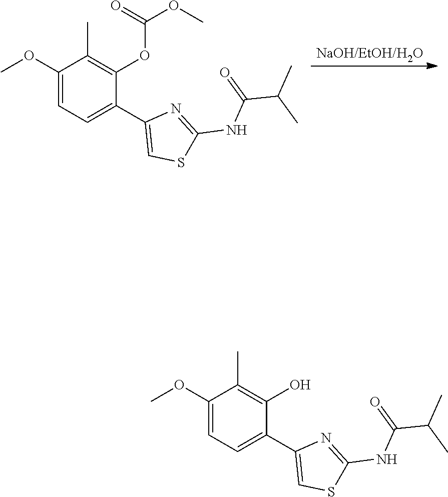

The examples below are intended to illustrate the present invention without limiting it. Unless otherwise stated, all quantitative data, fractions, and percentages are based on the weight and the total amount or on the total weight of the preparations.

### Example Formulations

### O/W Emulsions

| | Example formulations | | |
|---|---|---|---|
| | 1 | 2 | 3 |
| | % by weight | % by weight | % by weight |
| Stearic acid | 2.50 | 2.00 | 2.00 |
| Glyceryl stearate | 1.00 | 1.00 | 1.00 |
| C12-15 alkyl benzoate | 3.00 | 5.00 | 3.00 |
| Caprylic acid/capric | 2.50 | 2.50 | 2.00 |
| acid triglyceride | | | |
| Isopropyl palmitate | 2.00 | — | — |
| Cetyl stearyl alcohol | 3.00 | — | 2.00 |
| Cetyl alcohol | — | 2.00 | — |
| Stearyl alcohol | — | 2.00 | 1.00 |
| Dibutyl adipate | — | — | 1.50 |
| Cyclomethicone | 1.00 | 1.00 | 0.50 |
| Dicaprylyl carbonate | 2.00 | 2.00 | 2.00 |
| Dimethicone | 1.00 | — | 0.50 |
| Compound 1 | 0.1 | 0.125 | 0.15 |
| Glycerin | 5.00 | 7.00 | 5.00 |
| Ethylhexyl cocoate | — | — | 1.00 |
| Butylene glycol | — | — | 2.0 |
| Carbomer | 0.15 | 0.10 | 0.15 |
| Carrageenan | 0.10 | — | 0.10 |
| Xanthan gum | — | — | 0.10 |
| Acrylates/C10-30 Alkyl | — | 0.10 | — |
| Acrylate Crosspolymer | | | |
| Trisodium EDTA | 0.20 | 0.20 | 0.20 |
| Tapioca starch | 1.50 | 1.00 | — |
| Polymethylsilsesquioxane | — | 1.00 | 1.00 |
| Aluminum starch | — | — | 1.00 |
| octenylsuccinate | | | |
| Distarch phosphate | 1.00 | 1.00 | — |
| Sodium hydroxide | as | as | as |
| | required | required | required |
| Water | to 100 | to 100 | to 100 |
| | Example formulations | | |
| | 4 | 5 | 6 |
| | % by weight | % by weight | % by weight |
| Stearic acid | 2.50 | 2.00 | 2.00 |
| Glyceryl stearate | 1.00 | 1.00 | 1.00 |
| C12-15 alkyl benzoate | 3.00 | 5.00 | 3.00 |
| Caprylic acid/capric | 2.50 | 2.50 | 2.00 |
| acid triglyceride | | | |
| Isopropyl palmitate | 2.00 | — | — |
| Cetyl stearyl alcohol | 3.00 | — | 2.00 |
| Cetyl alcohol | — | 2.00 | — |
| Stearyl alcohol | — | 2.00 | 1.00 |
| Dibutyl adipate | — | — | 1.50 |
| Cyclomethicone | 1.00 | 1.00 | 0.50 |
| Dicaprylyl carbonate | 2.00 | 2.00 | 2.00 |
| Dimethicone | 1.00 | — | 0.50 |
| Compound 12 | 0.2 | 0.15 | 0.1 |
| Glycerin | 5.00 | 7.00 | 5.00 |
| Ethylhexyl cocoate | — | — | 1.00 |
| Butylene glycol | — | — | 2.0 |
| Carbomer | 0.15 | 0.10 | 0.15 |
| Carrageenan | 0.10 | — | 0.10 |
| Xanthan gum | — | — | 0.10 |
| Acrylates/C10-30 Alkyl | — | 0.10 | — |
| Acrylate Crosspolymer | | | |
| Trisodium EDTA | 0.20 | 0.20 | 0.20 |
| Tapioca starch | 1.50 | 1.00 | — |
| Polymethylsilsesquioxane | — | 1.00 | 1.00 |
| Aluminum starch | — | — | 1.00 |
| octenylsuccinate | | | |
| Distarch phosphate | 1.00 | 1.00 | — |
| Sodium hydroxide | as | as | as |
| | required | required | required |
| Water | to 100 | to 100 | to 100 |

### W/O Emulsions

Example formulations

| | 7 | 8 |
|---|---|---|
| Chemical/INCI name | % by weight | % by weight |
| Polyglyceryl-3 diisostearate | 1.50 | 1.50 |
| PEG-40 sorbitan perisostearate | 2.50 | 2.50 |
| Lanolin alcohol | 0.50 | 0.50 |
| Paraffinum Liquidum (mineral oil) | 8.00 | 8.00 |
| Cera Microcrystallina | 2.50 | 2.50 |
| Cyclomethicone | 4.00 | 4.00 |
| Isohexadecane | 2.00 | 2.00 |
| Isopropyl palmitate | 5.00 | 5.00 |
| Iodopropynyl butylcarbamate | — | 0.10 |
| Compound 1 | 0.20 | 0.10 |
| Glycerin | 7 | 7 |
| Perfume | as required | as required |
| Water | to 100 | to 100 |

Example formulations

| | 9 | 10 |
|---|---|---|
| Chemical/INCI name | % by weight | % by weight |
| Polyglyceryl-3 diisostearate | 1.50 | 1.50 |
| PEG-40 sorbitan perisostearate | 2.50 | 2.50 |
| Lanolin alcohol | 0.50 | 0.50 |
| Paraffinum Liquidum (mineral oil) | 8.00 | 8.00 |
| Cera Microcrystallina | 2.50 | 2.50 |
| Cyclomethicone | 4.00 | 4.00 |
| Isohexadecane | 2.00 | 2.00 |
| Isopropyl palmitate | 5.00 | 5.00 |
| Iodopropynyl butylcarbamate | — | 0.10 |
| Compound 12 | 0.10 | 0.15 |
| Glycerin | 7 | 7 |
| Perfume | as required | as required |
| Water | to 100 | to 100 |

### Aqueous-Ethanolic Solution for e.g., Pen/Applicator/Brush Application

| | Example formulation |
|---|---|
| | 11 |
| | % by weight |
| Sodium polyacrylates | 1.00 |
| Alcohol Denat. + Aqua | 44.40 |
| Glycerin | 10.00 |
| Compound 1 | 0.20 |
| Water | 44.40 |

| | Example formulation |
|---|---|
| | 12 |
| | % by weight |
| Sodium polyacrylates | 1.00 |
| Alcohol Denat. + Aqua | 44.40 |
| Glycerin | 10.00 |
| Compound 12 | 0.20 |
| Water | 44.40 |

1.-7. (canceled)

8. An isobutyramide of general formula: 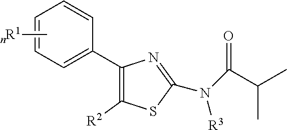
wherein
n: is an integer from 1 to 5 which indicates the number of radicals R¹,
R¹: independently represents OH, CH₃, O—CH₃, O—(CO)—CH₃,
R²: represents H or C₁-C₄ alkyl,
R³: represents H or CH₃,
the isobutyramide of formula 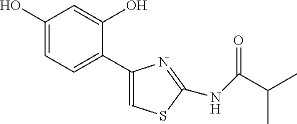 [SMILES: CC(C)C(=O)Nc1nc(-c2ccc(O)cc2O)cs1]
being excluded.

9. The isobutyramide of claim 8, i.e., one of the following compounds:

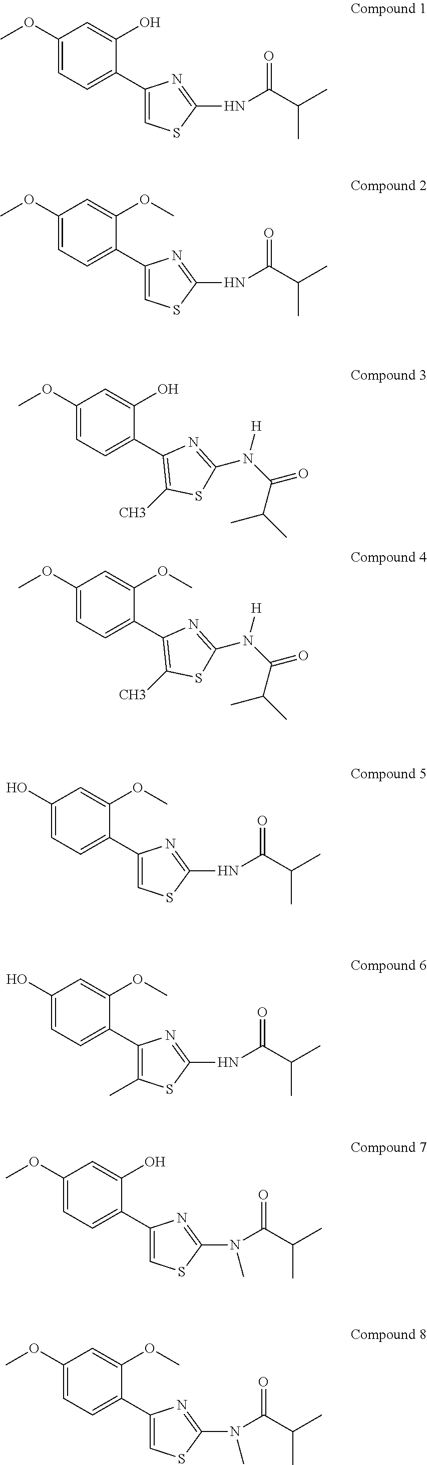

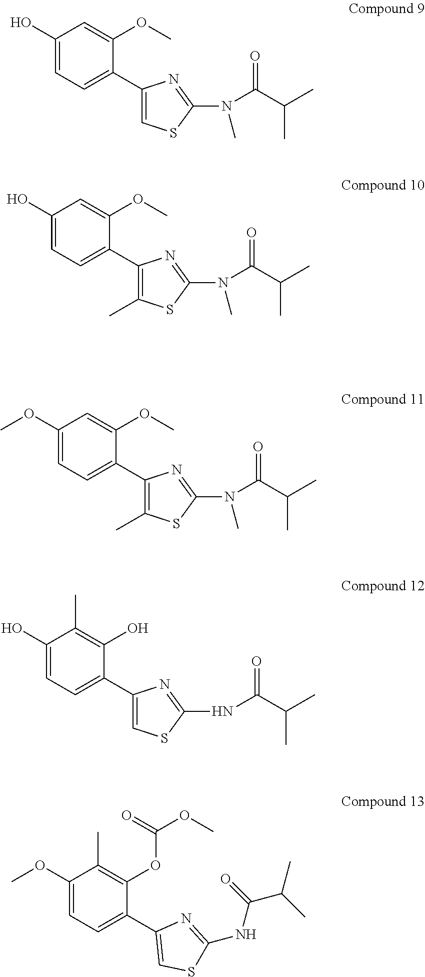

10. A cosmetic or dermatological preparation, wherein the preparation comprises one or more isobutyramides according to claim 8.

11. A cosmetic or dermatological preparation, wherein the preparation comprises one or more isobutyramides according to claim 9.

12. The preparation of claim 10, wherein the preparation comprises from 0.000001% to 10.0% by weight of the one or more isobutyramides, based on a total weight of the preparation.

13. The preparation of claim 12, wherein the preparation comprises from 0.0001% to 3.0% by weight of the one or more isobutyramides.

14. The preparation of claim 12, wherein the preparation comprises from 0.001% to 1% by weight of the one or more isobutyramides.

15. The preparation of claim 10, wherein the preparation is suitable for topical application to human skin.

16. The preparation of claim 10, wherein the preparation is present as an aqueous or aqueous/ethanolic solution.

17. The preparation of claim 10, wherein the preparation is present as an emulsion.

18. The preparation of claim 10, wherein the preparation is capable of prophylaxis and treatment of sensitive skin, itching, dry skin and/or inflammatory phenomena in human skin.

19. The preparation of claim 10, wherein the preparation is capable of reducing a production of sebum or a use of cyclodextrins to produce preparations for eliminating sebum.

20. The preparation of claim 10, wherein the preparation is capable of preventing a formation of comedones and/or acne.

21. The preparation of claim 10, wherein the preparation is capable of preventing or eliminating seborrheic phenomena.

22. The preparation of claim 10, wherein the preparation is capable of preventing or eliminating greasy hair.

23. The preparation of claim 10, wherein the preparation is capable of preventing or eliminating dandruff.

24. A method for the prophylaxis and treatment of sensitive skin, itching, dry skin and/or inflammatory phenomena in human skin, wherein the method comprises applying to skin in need thereof the preparation of claim 10.

25. A method for the prophylaxis and treatment of or for reducing the production of sebum or the use of cyclodextrins for producing preparations for eliminating sebum, wherein the method comprises employing an isobutyramide of claim 8 in making a cosmetic or dermatological preparation for topical application to skin.

26. A method of preventing the formation of comedones and/or acne, wherein the method comprises applying to skin in need thereof a preparation of claim 10.

27. A method of preventing or eliminating seborrheic phenomena, wherein the method comprises applying to skin in need thereof a preparation of claim 10.
